(19) United States
(12) Patent Application Publication
(10) Pub. No.: US 2024/0277601 A1
(43) Pub. Date: Aug. 22, 2024

(54) NOVEL COSMETIC COMPOSITION IN GRANULAR FORM

(71) Applicant: PIERRE FABRE DERMO-COSMETIQUE, Boulogne (FR)

(72) Inventors: Laetitia LEMERCIER, Flourens (FR); Philippe TRANNOY, Toulouse (FR)

(21) Appl. No.: 18/026,912

(22) PCT Filed: Sep. 30, 2021
(86) PCT No.: PCT/EP2021/077019

§ 371 Date: Mar. 17, 2023

(30) Foreign Application Priority Data
Oct. 1, 2020 (FR) 2010045

(51) Int. Cl. A61K 8/96; A61K 8/26; A61K 8/46; A61K 8/73; A61K 8/9794; A61Q 5/00; A61Q 5/02

(52) U.S. Cl. CPC A61K 8/965; A61K 8/26; A61K 8/466; A61K 8/73; A61K 8/9794; A61Q 5/006; A61Q 5/02; A61K 2800/48; A61K 2800/651

(57) ABSTRACT

A cosmetic composition which is in dry form and suitable for being topically applied to the skin and/or keratin fibres, and which includes at least 50% by volume of disintegratable granules containing a mixture of at least one anionic surfactant, at least one absorbent agent and at least one thickening agent chosen from gums of plant, microbial and/or synthetic origin, the total amount of the thickening agent(s) in the composition being between 1 and 4% by weight relative to the total weight of the composition.

### CROSS REFERENCE TO RELATED APPLICATIONS

The application is a National Stage of International Application No. PCT/EP2021/077019, having an International Filing Date of 30 Sep. 2021, which designated the United States of America, and which International Application was publishing under PCT Article 21(2) as WO Publication No. 2022/069671 A1, which claims priority from and the benefit of French Patent Application No. 2010045, filed on 1 Oct. 2020, the disclosures of which are incorporated herein by reference in their entireties.

The present disclosure relates to the field of care and hygiene of the skin and/or of keratin fibres, in particular human, especially hair.

More specifically, the present disclosure relates to a cosmetic composition in dry form of the type suitable for being topically applied to the skin and/or keratin fibres, especially human, as well as to a disintegratable granule suitable for such an application and a method for preparing such a disintegratable granule. The disclosure also relates to a method for washing and/or cosmetically treating keratin fibres, especially human, as well as a method for washing and/or cosmetically treating the skin, especially human, these two methods using a cosmetic composition according to the disclosure. The present disclosure also relates to the cosmetic use of such a composition for exfoliating the skin of an individual's face and/or body.

### PRIOR ART

In the field of washing keratin fibres, in particular hair on the body and head, numerous cosmetic products are known. These products are generally intended to cleanse the keratin fibres while providing them with good cosmetic properties. Conventional products for cleansing keratin fibres, such as shower gels and shampoos, are most frequently in the form of more or less thickened liquids. However, these products can prove to be difficult to measure out: the more liquid they are, the more they tend to flow between the fingers, making them difficult to measure out and generating waste, and the more they can leak out of their packaging. For these reasons, it may be advantageous to use foaming products in dry form, which contain little or no water.

Dry shampoos have existed for many years, either in powder form, or in spray form. They make it possible to remove excess sebum quickly without wetting the hair. They act by capturing sebum by absorption thanks to powders chosen for the sebum-absorbing properties thereof. The powders used can be of mineral, organic or synthetic origin, and can particularly be wheat, rice and corn starch derivatives. In practice, commercially available dry shampoos are not entirely satisfactory. In particular, the clean sensation perceived by users does not last all day long, particularly after physical exercise. Moreover, the styling properties thereof, such as adding volume to the hair and lifting the roots, are generally very limited. Furthermore, they do not enable effective removal of excess sebum present on the scalp and the annoyances caused such as irritations or odours.

In addition, the stability of shampoos in the form of sprays comprising powders is difficult to obtain.

Cosmetic compositions in dry powder form, whether they are intended to be applied to the skin or to keratin fibres, do not make it possible for their part to obtain foaming initiation at the desired time, and they do not always make it possible to obtain a sufficient abundance of foam. Furthermore, when using pulverulent powders, users may have difficulty removing them on rinsing and residues may remain on the skin or the hair.

Finally, dry compositions in powder form are difficult to handle, both during the manufacture and the use thereof. An example of such a dry composition in powder form is described in the document CN105687054.

Solid cosmetic compositions in granule form, easier to handle than powders, have been proposed by the prior art. Mention can be made in this regard of the document FR 3 030 269, which describes solid anhydrous cosmetic compositions in the form of particles comprising at least 30% by weight of anionic surfactant(s), at least 5% by weight of amphoteric surfactant(s) and at least 10% by weight of filler(s); or the document FR 3 068 243 which describes solid cosmetic compositions in the form of granules containing an anionic surfactant chosen from isethionic acid derivatives, at least one anionic surfactant chosen from glutamic acid derivatives, at least one amphoteric surfactant and at least one 20% by weight of fillers. These compositions in granule form generate, in contact with water, a very abundant foam very rapidly, this foam furthermore not being stable over time. They particularly have the drawback of requiring a large amount of rinsing water.

Therefore, there is a need to develop novel care and hygiene compositions for the skin and/or keratin fibres, such as hair on the body and head, which are easy to handle, which make it possible to form, in contact with water, well-controlled foam, in terms of foam formation rate, to provide the best possible convenience of use for the user, and in terms of the amount of foam formed, so as to limit the amount of rinsing water required, while having an at least equally good, or even better, washing and/or cosmetic care performance, compared to that of the dry compositions proposed by the prior art, i.e. to develop cosmetic compositions offering optimal cleansing activity with foaming initiation with an optimal rate and an optimal abundance of foam, and good cosmetic properties, in particular, in the case of cosmetic hair treatment, added volume to the hair, with a soft sensation and a long-lasting clean sensation. The present disclosure is intended to achieve these aims. A further aim of the disclosure is that the foam formed upon contacting the composition with water be sufficiently stable throughout the normal duration of use of such a composition by consumers, in order to optimise the cleansing effectiveness of the composition. Typically, for a shampoo for example, this duration is approximately 5 minutes.

### SUMMARY

Surprisingly and unexpectedly, the inventors discovered that cosmetic washing compositions in dry form comprising at least 50% by volume of disintegratable granules, of specific constitution, generate in contact with water a sufficient amount of foam that is sufficiently stable over time to ensure good washing effectiveness, without requiring an excessive amount of water for rinsing, the disintegration of the granules being furthermore advantageously progressive, so that the foam generation is delayed slightly over time and an initial exfoliating effect is obtained. These advantageous results are associated with the presence in the disintegratable granules, besides at least one anionic surfactant and at least one absorbent agent, of one or more thickening agents chosen from gums, in a total amount between 1 and 4% by weight relative to the total weight of the composition. Such compositions provide the cleansing properties expected of a cosmetic composition for washing the skin and/or keratin fibres, especially of a shampoo or a mask, as well as, in the latter case, advantageous styling properties such as adding volume and body to the hair with a long-lasting soft, clean and fresh sensation; but also scalp-cleansing properties. These novel cosmetic compositions are also of particularly interest owing to the easy handling thereof during the manufacture and packaging thereof, the easy use thereof, especially in that they reduce, compared to conventional powder compositions, the presence of dust during the use thereof. They are furthermore particularly advantageous in that they enable, by the control of the characteristics of the granules, especially the size thereof, an even finer control of the foam formation rate by contacting the anionic surfactant with water, this rate being easily adaptable according to the specific application sought.

Moreover, these cosmetic compositions according to the disclosure, by the presence of the granules and the slow disintegration rate thereof, are endowed with an exfoliating activity due to the physical characteristics of these granules, which renders them particularly advantageous for cosmetic cleansing and care applications for the skin and/or keratin fibres, especially human.

According to a first aspect, the disclosure thus relates to a cosmetic composition which is in dry form, suitable for being topically applied to the skin and/or keratin fibres, comprising at least 50% by volume of disintegratable granules having a size, measured by screening, between 150 and 2000 μm and containing a mixture of: —at least one anionic surfactant,
at least one absorbent agent chosen from clays, ceramic beads, calcium carbonate, titanium oxides, kaolin, aluminium silicates, starches, celluloses, sodium carbonate, Nylon microspheres and plant-based powders,
and at least one thickening agent chosen from gums of plant, microbial and/or synthetic origin,
   the total amount in the composition of this or these thickening agent(s) chosen from gums of plant, microbial and/or synthetic origin being between 1 and 4% by weight relative to the total weight of the composition, preferably between 2 and 4% by weight relative to the total weight of the composition.

Preferably, the total amount of this or these thickening agent(s) chosen from gums of plant, microbial and/or synthetic origin, present in the composition, is contained in the disintegratable granules.

According to a second aspect, the disclosure relates to a method for washing and/or non-therapeutically cosmetically treating keratin fibres, comprising a step of applying, to the damp keratin fibres, such a cosmetic composition in dry form comprising at least 50% by volume of disintegratable granules, this application step being optionally followed by massaging to develop foam, followed by an optional application time of 0 to 5 minutes, followed by rinsing of the keratin fibres with water.

According to a third aspect, the disclosure relates to a method for washing and/or non-therapeutically cosmetically treating the skin, comprising a step of applying, to the damp skin, such a cosmetic composition in dry form comprising at least 50% by volume of disintegratable granules, this application step being optionally followed by massaging to develop foam, followed by an optional application time of 0 to 5 minutes, followed by rinsing of the skin with water.

According to a fourth aspect, the disclosure relates to the non-therapeutic cosmetic use, for exfoliating the skin of an individual's face and/or body, of such a cosmetic composition in dry form comprising at least 50% by volume of disintegratable granules.

According to a fifth aspect, the disclosure relates to a disintegratable granule which is suitable for being topically applied to an individual's skin and/or hair, suitable for being contained in the composition according to the disclosure, as described above, this disintegratable granule having a size, measured by screening, between 150 and 2000 μm and containing a mixture of:
at least one anionic surfactant,
at least one thickening agent chosen from gums of plant, microbial and/or synthetic origin,
and at least one absorbent agent chosen from clays, ceramic beads, calcium carbonate, titanium oxides, kaolin, aluminium silicates, starches, celluloses, sodium carbonate, Nylon microspheres and plant-based powders,
   the total amount in the composition of this or these thickening agent(s) chosen from gums of plant, microbial and/or synthetic origin, in this granule, being between 1 and 4% by weight, preferably between 2 and 4% by weight, relative to the total weight of the granule.

A further aspect of the disclosure relates to a method for preparing such a disintegratable granule, this method comprising successive steps of:
preparing a mixture containing said at least one anionic surfactant, said at least one absorbent agent and said at least one thickening agent, in an amount between 1 and 4% by weight, preferably between 2 and 4% by weight, relative to the total weight of the granule,
adding a wetting liquid to this mixture,
forming a granule,
and drying this granule.

### Definitions

In the present disclosure, “granule” denotes, conventionally per se, a preparation consisting of dry, solid grains each formed by a cohesive agglomerate of powder particles, the granules being typically obtained by a granulation method.

“Disintegratable” denotes an ability of a solid entity to break down, at a more or less high speed, in the presence of water and/or under the effect of a mechanical friction exerted thereon.

“Powder” denotes, according to the present disclosure, conventionally per se, a dosage form consisting of free, dry, solid particles, each being formed of a single compound.

“Dry form” denotes, according to the present disclosure, a cosmetic composition which does not comprise water/is anhydrous, or a composition comprising a water content less than or equal to 5% by weight, preferably less than or equal to 2% by weight, preferably less than or equal to 1% by weight, and more preferably less than or equal to 0.5% by weight, relative to the total weight of the composition, or a cosmetic composition which does not contain added water, i.e., any water present in the cosmetic composition according to the disclosure is none other than bound water, such as salt crystallisation water, or water traces absorbed by the raw materials used in producing the cosmetic composition, or else residual water from a wet-process granulation method optionally used for granule formation.

“Mask” denotes, according to the disclosure, a type of facial and/or scalp treatment consisting of the prolonged application of a cosmetic product to the face, scalp and/or hair. This mask is a product intended to be applied to damp/wet skin/hair and then rinsed after an application time.

“Topical application” denotes, according to the present disclosure, an application to the skin (including the scalp), mucosa and/or keratin fibres.

“Keratin fibres” denotes, according to the present disclosure, the hair, eyelashes, eyebrows and body hair, preferably beard hair. According to the disclosure, the beard includes the moustache.

“Cosmetically acceptable” denotes, according to the present disclosure, that which is useful in the preparation of a cosmetic composition, which is generally safe, non-toxic and neither biologically nor otherwise undesirable, and which is acceptable for cosmetic use, particularly by topical application to the skin (including the scalp), mucosa and keratin fibres.

“Anionic surfactant” denotes, according to the present disclosure, a surfactant only including anionic groups as ionic or ionisable groups. In the present disclosure, an entity is described as “anionic” when it has at least one permanent negative charge or when it can be ionised to a negatively charged entity, especially under the conditions of use of the composition of the disclosure (medium, pH, for example), and it has no cationic charge.

“Absorbent agent” denotes an agent capable of absorbing a fluid, for example sebum.

In the present description, and unless specified otherwise, the expression “at least one” is equivalent to the expression “one or more” and can be replaced thereby. The expression “between” is equivalent to the expression “ranging from . . . to . . . ” and can be replaced thereby, and implies that the bounds are included.

### BRIEF DESCRIPTION OF THE FIGURES

FIG. 1 shows the sensory analysis profiles of a cosmetic hair care composition according to the disclosure C1 and a comparative composition not according to the disclosure, devoid of gum, Comp1;

FIG. 2 shows a graph illustrating, as a function of time, the peak thickness measured during a Turbiscan® physical stability analysis test after mixing in water and stirring to induce foam formation, for a cosmetic hair care composition according to the disclosure C1 and comparative compositions not according to the disclosure, devoid of gum, Comp1 and Comp2;

and FIG. 3 shows a graph illustrating, as a function of time, the mean backscatter value measured during a Turbiscan® physical stability analysis test after mixing in water and stirring to induce foam formation, for a cosmetic hair care composition according to the disclosure C1 and a comparative composition not according to the disclosure, devoid of gum, Comp1.

### DETAILED DESCRIPTION

According to a first aspect, the disclosure relates to a cosmetic composition which is in dry form, more specifically in the form of a divided solid, and suitable for being topically applied to the skin and/or keratin fibres, in particular of a human individual. This cosmetic composition comprises at least 50% by volume, relative to the total volume of the composition, of disintegratable granules containing a mixture of at least one anionic surfactant, at least one thickening agent and at least one absorbent agent.

This means that each individual granule contains, in a mixture, at least one anionic surfactant, at least one thickening agent and at least one absorbent agent.

The use of granules, in a majority amount by volume in the composition, especially makes it possible, particularly advantageously, to increase the flow capacity of the composition, which in particular facilitates the removal thereof from a sachet wherein it is packaged. It also facilitates the handling of the composition during the manufacture and packaging thereof, and during the use thereof, and reduces the dust generation by the composition. The solid form of the composition facilitates the storage and the transport thereof.

These advantages are obtained without penalising the washing and cosmetic treatment effectiveness of the composition, or even improving this effectiveness.

Firstly, it is observed that the granule form makes it possible to ensure a homogeneous and uniform mixture of the constituents of the granules, in particular of the anionic surfactant, the thickening agent and the absorbent agent, which improves the washing and care performances of the cosmetic composition according to the disclosure. These performances are particularly good, in respect of cleansing of the target area, the clean sensation perceived by the user, and the cosmetic effects obtained, for example the addition of volume to the hair in the case of shampoos or hair masks. The action of the specific constituents of the granules which are the anionic surfactant, the thickening agent and the absorbent agent, is thus not only in no way prevented, but, on the contrary, it is particularly substantial.

The anionic surfactant ensures in particular a substantial washing effect, and foam formation in contact with water.

The absorbent agent advantageously makes it possible, for its part, to absorb any oily fluid present on the skin or keratin fibres, such as lipophilic dirt and in particular excess sebum. During the manufacture of the granules, it furthermore facilitates granulation by acting as binder. It can also make it possible to incorporate fluid substances, that it is capable of absorbing, inside the granules.

Furthermore, a suitable adjustment of the physical characteristics of the granules, especially of the size and shape thereof, in association with the presence therein of one or more thickening agents chosen from gums of plant, microbial and/or synthetic origin, in the specific concentration range recommended by the disclosure, makes it to possible to control the disintegration rate thereof, in particular in the presence of water, and hence the duration of the exfoliating effect thereof, and the foam formation rate in the presence of water.

The granules used in the composition according to the disclosure advantageously have a somewhat slow disintegration rate. It is understood thereby that the granule does not disintegrate too quickly, in the presence of water and/or when repeated mechanical friction is exerted thereon, for example by the user's hand.

The time to obtain complete disintegration of the granules according to the disclosure, placed in the presence of water and/or rubbed, is preferably greater than 30 seconds, preferably greater than 1 minute, preferably greater than 2 minutes, and more preferably greater than 3 minutes. This time is furthermore preferably less than 5 minutes.

This disintegration time of a granule according to the disclosure can for example be determined by placing the composition according to the disclosure in water, for example at a rate of 3 g of composition in 100 ml of water, at 25° C., under stirring, for example at 200 rpm, and by determining the time required for the granules contained in the composition to completely disappear.

Preferably, the granules used in the composition according to the disclosure are such that, when they undergo the above protocol, the partial presence of granules after 3 minutes, and an absence of granules after 4 minutes, are observed.

The granules used in the composition according to the disclosure can be obtained both with a dry-process granulation method and with a wet granulation method.

In specific embodiments of the disclosure, the cosmetic composition comprises at least 75% by volume, relative to the total volume, of said granules.

The composition can contain, besides the granules, any other cosmetically acceptable ingredient, such as a filler, a perfume, etc., each of these ingredients obviously being in the form of a divided solid or being absorbed on a divided solid.

The granules used in the composition can also contain any other cosmetically acceptable ingredient.

The ingredients contained in the composition according to the disclosure are preferably of natural origin, thus meeting a very strong demand for formulations containing ingredients of natural origin which has characterized the cosmetic market for several years. Indeed, consumers seek formulations free from chemical substances, preferring ingredients of natural origin, renowned for their superior tolerance and affinity with the skin, and which are more respectful of the environment.

In specific embodiments of the disclosure, the cosmetic composition comprises one or more cosmetically active agents, this or these agent(s) optionally being present in the granules and/or outside the granules. By way of cosmetically active agents, mention can be made of a nettle root extract for the purifying and seboregulating action thereof or a dry Alpinia galanga extract, having an anti-dandruff action.

The cosmetic composition according to the disclosure can furthermore comply with one or more of the features described hereinafter, used separately or in each of the technically feasible combinations thereof.

Hereinafter in the description, the term “granule” is used to denote the disintegratable granules contained at at least 50% by volume in the composition according to the disclosure and containing a mixture of at least one anionic surfactant, at least one thickening agent as defined above and at least one absorbent agent as defined above, it being understood that the composition according to the disclosure can also contain other types of granules of another composition, which will not be specifically described in the present description.

According to specific embodiments, the cosmetic composition comprises at least 60% by volume, relative to the total volume of the composition, of the granules.

According to specific embodiments, the cosmetic composition comprises at least 70% by volume, relative to the total volume of the composition, of the granules.

According to specific embodiments, the cosmetic composition comprises at least 75% by volume, relative to the total volume of the composition, of the granules.

According to specific embodiments, the cosmetic composition comprises at least 80% by volume, relative to the total volume of the composition, of the granules.

In specific embodiments of the disclosure, the granules have a size, measured by screening, optionally manual, between 315 and 2000 μm, and even more advantageously between 315 and 1250 μm.

In other specific embodiments of the disclosure, the size of the granules according to the disclosure is between 300 and 2000 μm.

The size of the granules can otherwise be determined via a mechanical calibrator, and by laser granulometry.

When the size is determined by screening, the size is taken in the smallest dimension of the granule.

Preferably, the granules according to the disclosure are in the form of small divided objects formed of aggregated solid particles, of variable shapes and sizes. They can be of regular or irregular shape. They can in particular be of spherical, square, rectangular, or elongated shape, such as rods.

The granules according to the disclosure are such that they disintegrate slowly. This feature enables the granules according to the disclosure to develop the exfoliating activity thereof in the first minutes after being applied to the skin or keratin fibres, before the complete disintegration thereof.

In specific implementations of the disclosure, the composition contains an amount of anionic surfactant(s) between 1 and 50% by weight, preferably between 5 and 40% by weight, relative to the total weight of the composition. The surfactant(s) can be present only inside the granules, or also be present in the composition outside the granules, for example in pulverulent powder form.

The granules according to the disclosure comprise at least one, optionally, several, anionic surfactant(s), which advantageously provide(s) the composition with the cleansing, and foaming, property. This or these anionic surfactant(s) are preferably dispersible in water. This or these anionic surfactant(s) can be chosen for example from carboxylates, sulphonates, phosphates, isethionates, sarcosinates, glutamates, lactilates, taurates, fatty acid salts, uronic galactoside salts, carboxylic ether acid salts and mixtures thereof.

According to embodiments of the disclosure, a surfactant particularly suitable for the disclosure can be chosen from isethionic acid derivatives and the salts thereof.

This or these anionic surfactant(s) can for example be chosen from acyl isethionic acids, the salts thereof (isethionates) and the mixtures thereof, preferably from acyl isethionic acid salts wherein the hydrocarbon chain R₁ of the acyl group R₁C═O, is linear or branched, saturated or unsaturated, and comprises from 8 to 30 carbon atoms, preferably from 10 to 20 carbon atoms, preferably from 12 to 18 carbon atoms. The acyl group can especially be chosen from the lauroyl, myristoyl, palmitoyl, stearoyl, olivoyl, cocoyl, oleoyl groups and the mixtures thereof.

“Salts thereof” denotes that the hydrogen atom of the acid function of the isethionic acid is replaced by a cation M⁺, for example chosen from ions of alkali metals such as Na, Li, K, preferably Na or K, from ions of alkaline earth metals such as Mg, ammonium groups and the mixtures thereof.

By way of examples of isethionic acid derivatives suitable for use within the scope of the disclosure, mention can be made for example of the compound having the INCI name sodium lauroyl methyl isethionate such as the references Iselux® LQ-CLR, Iselux® LQ-CLR-SB, or the sodium lauroyl methyl isethionate/sodium methyl isethionate mixture such as Iselux® proposed by Innospec. Mention can also be made of the compound bearing the INCI name sodium cocoyl isethionate such as the references Hostapon® SCI marketed by Clariant or Pureact I-85EC marketed by Innospec Active Chemicals. Mention can also be made of sodium cocoyl-isethionate marketed under the trade name Jordapon® CI by BASF. A preferred anionic surfactant in the context of the disclosure is sodium cocoyl-isethionate as marketed by Akzo Nobel under the trade name Elfan® AT84G.

According to preferred embodiments of the disclosure, the granules according to the disclosure comprise a single anionic surfactant, preferably derived from isethionic acid.

Preferably, the cosmetic composition according to the disclosure is devoid of sulphate-containing anionic surfactant. The expression “sulphate-containing anionic surfactant” denotes an anionic surfactant comprising at least one sulphate function (—OSO₃H or —OSO₃⁻), and optionally comprising one or more other functions derived from acids, such as carboxylic acid or carboxylate functions (—COOH or —COO⁻), sulphonate functions (—SO₃H or —SO₃⁻) and/or phosphate functions.

The cosmetic composition according to the disclosure can contain, in addition to the anionic surfactant or instead of the anionic surfactant, in and/or outside the granules, one or more non-ionic surfactants. This or these non-ionic surfactant(s) can for example be chosen from alkyl polyglucosides.

In specific embodiments of the disclosure, the total amount of surfactants contained in the composition, in particular in the disintegratable granules, is between 1 and 50% by weight relative to the total weight of the composition. Such a feature, associated with the presence in the granules of at least one thickening agent chosen from gums, the total amount of gums in the composition being between 1 and 4% by weight relative to the total weight of the composition, advantageously makes it possible to form, in contact with water, a foam of optimal abundance with respect to the washing performances of the composition and the ease of rinsing.

The granules according to the disclosure comprise at least one absorbent agent, which particularly makes it possible to remove excess sebum on the skin and/or keratin fibres. This absorbent agent can be of mineral or organic origin.

According to embodiments of the disclosure, the absorbent agent is dispersible in water.

By way of absorbent agent of mineral origin, mention can be made of clays, ceramic beads, calcium carbonate, titanium oxides, kaolin or aluminum silicate.

By way of absorbent agent of organic origin, mention can be made of starches, celluloses such as cellulose beads, sodium carbonate, Nylon microspheres, micronised or non-micronised plant-based powders such as fruit powders or bamboo powders, or rice grain husk powder, and the mixtures thereof.

The starch derivatives used in the present disclosure can be obtained from a plant source such as cereals, tubers, roots, vegetables, and fruits.

Preferably, the absorbent agent is chosen from starches.

The starches suitable for use in the present disclosure are, for example corn starch, potato starch, pea starch, rice starch, oat starch, barley starch, tapioca starch, wheat starch, cassava starch, and sorghum.

These starches can be modified or not. A modified starch is a starch which has been modified by methods known to a person skilled in the art, such as for example esterification, etherification, oxidation, acid hydrolysis, cross-linking or enzyme conversion.

According to specific embodiments of the disclosure, the cosmetic composition according to the disclosure contains at least one absorbent agent other than silica and talc.

According to specific embodiments of the disclosure, the cosmetic composition contains at least one absorbent agent in the form of white powder, the elementary particle size whereof varies from 15 to 100 μm, this absorbent agent being present in and/or outside the granules.

According to embodiments of the disclosure, the granules contain at least, in and/or outside the granules, three absorbent agents chosen from starches and clays.

According to embodiments of the disclosure, the composition according to the disclosure contains at least, in and/or outside the granules, two absorbent agents chosen from starches and clays.

According to other embodiments of the disclosure, the composition according to the disclosure only contains a single absorbent agent.

The absorbent agent(s) can be present in the composition according to the disclosure in an amount ranging from 1 to 80% by weight, preferably from 2 to 60% by weight and preferentially from 20 to 60% by weight, relative to the total weight of the composition.

In specific embodiments of the disclosure, the granules further comprise at least one clay.

The granules according to the disclosure comprise one or more thickening agents chosen from gums of plant origin, gums of microbial origin and/or gums of synthetic origin. The gums of plant origin are extracted from trees, leafy shrubs and algae. The gums of microbial origin are obtained by synthesis of these macromolecules by selected micro-organism strains under controlled conditions in fermenters. As for synthetic gums, they are obtained by modifying or deriving natural gums and by chemical synthesis.

The gums of plant origin can be chosen from gum arabic, ghatti gum, karaya gum, pectins, guar gum, carob bean gum, agar, alginates, carrageenans,

The gums of microbial origin can be chosen from scleroglucan gum, gellan gum, pullulan gum, Curdlan gum, xanthan gum, grifolan gum, lentinan gum, Schizophyllan gum, spirulinan gum and krestin gum.

The gums of synthetic origin can be chosen from cellulose derivatives.

According to embodiments of the disclosure, the composition according to the disclosure contains two thickening agents which are preferably chosen from gums of plant origin and/or microbial origin.

Preferably, particularly advantageous gums in the context of the present disclosure are gum arabic and/or xanthan gum.

The thickening agent(s) are present in the composition according to the disclosure in an amount ranging from 1 to 4% by weight, preferably from 2 to 4% by weight, and preferentially from 2 to 3% by weight, relative to the total weight of the composition. In the composition, they are preferably only contained in the disintegratable granules.

According to specific embodiments of the disclosure, the composition according to the disclosure further comprises an exfoliating agent, which is preferably present therein outside the granules, and which will enable exfoliation of the skin (including the scalp), by the mechanical action thereof. The exfoliating agent can be chosen from agents of mineral, plant, or organic origin. Thus, it is possible to use for example polyethylene beads or powder, nylon particles, polyvinyl chloride powder, pumice stone (INCI name: pumice), walnut husks, sawdust, wood flour, ground cork, glass beads, alumina (aluminium oxide) (INCI name: Alumina), sugar crystals, beads which melt when applied to the skin, such as for example, spheres based on mannitol and cellulose, capsules based on agar and spheres based on jojoba esters, and the mixtures thereof. This or these exfoliating agent(s) are in particular preferably not water-soluble.

According to embodiments of the disclosure, the cosmetic composition according to the disclosure is devoid of sulphate, in order to improve the tolerance thereof.

According to embodiments of the disclosure, the cosmetic composition according to the disclosure is devoid of silica, in order to improve the tolerance thereof.

According to embodiments of the disclosure, the cosmetic composition according to the disclosure is devoid of talc, in order to improve the tolerance thereof.

According to embodiments of the disclosure, the cosmetic composition according to the disclosure is devoid of sulphate, silica, and talc.

The cosmetic composition according to the disclosure can further contain one or more additives, which can be present in and/or outside the granules, and for example chosen from anionic, cationic, non-ionic, amphoteric polymers, conditioners or fixatives, perfumes, dyes, protective filters, acids, bases, nacres, glitter, etc.

These additives can be present in the composition according to the disclosure in an amount ranging from 0 to 20% by weight relative to the total weight of the composition.

The person skilled in the art will take care to choose these optional additives and the amounts thereof such that they do not impair the properties of the composition of the present disclosure.

In specific embodiments of the disclosure, the granules contain an amount of polyol(s), especially propylene glycol, less than 2% by weight relative to the total weight of the composition, preferably less than 1% by weight and preferentially less than 0.5% by weight, relative to the total weight of the composition.

According to embodiments of the disclosure, the composition according to the disclosure is in a form adapted for a single topical application. The cosmetic composition according to the disclosure can especially be packaged in single-dose form, for example in a sachet.

According to embodiments of the disclosure, the cosmetic composition according to the disclosure is packaged in a sachet of 2 to 6 g, preferably, of 2 to 4 g, and more preferably in a 3 g sachet.

The cosmetic composition according to the disclosure can form a product for cleansing the skin, of the body and/or the face, the scalp and/or the keratin fibres, preferably the hair. It can for example be a shampoo, a shower gel, a hair or skin mask, etc.

It can also be an exfoliation product, i.e. a product which serves to remove dead cells from the epidermis, the skin (including the scalp) thus being smoothed, and cell renewal therefore being promoted. The exfoliating action thereof is especially great when, according to specific embodiments of the disclosure, it contains an exfoliating agent, outside the granules.

According to a second aspect, the disclosure relates to a method for washing and/or non-therapeutically cosmetically treating keratin fibres, especially human, and in particular hair on the body and head. This method includes a step of applying, to damp keratin fibres, a cosmetic composition according to the disclosure, as defined above, followed optionally by massaging to develop the foam, by contact of water with the anionic surfactant contained in the granules of the composition, followed by an optional application time that can preferably last up to 5 minutes, then finally rinsing of said keratin materials with water.

According to specific implementations of the disclosure, foam is developed prior to the step of applying the composition to the keratin fibres, by pouring the composition according to the disclosure into the hand and then adding water, the foaming composition thus obtained then being applied to the scalp and/or hair for washing, followed by a rinsing step after an optional application time.

According to a further aspect, the disclosure relates to a method for washing and/or non-therapeutically cosmetically treating the skin, especially human, this method including a step of applying, to damp skin, a cosmetic composition according to the disclosure, as defined above, followed optionally by massaging to develop the foam, by contact of water with the anionic surfactant contained in the granules of the composition, followed by an optional application time that can preferably last up to 5 minutes, then finally rinsing of the skin with water.

Here again, foam can be developed prior to the step of applying the composition to the skin, by pouring the composition according to the disclosure into the hand and then adding water, the foaming composition thus obtained then being applied to the skin for washing, followed by a rinsing step after an optional application time.

A further aspect of the disclosure relates to the non-therapeutic cosmetic use, for exfoliating the skin of the face and/or body of an individual, in particular human, of a cosmetic composition according to the disclosure, as defined above.

According to a further aspect, the present disclosure relates to a disintegratable granule intended to be used in a cosmetic composition, suitable for being topically applied to the skin and/or keratin fibres, especially the hair, of an individual, particularly a human individual, this granule having a size, measured by screening, between 150 and 2000 μm and containing a mixture of:
at least one anionic surfactant,
at least one thickening agent chosen from gums of plant, microbial and/or synthetic origin,
and at least one absorbent agent chosen from clays, ceramic beads, calcium carbonate, titanium oxides, kaolin, aluminium silicates, starches, celluloses, sodium carbonate, Nylon microspheres and plant-based powders,
   the total amount of the thickening agent(s) chosen from the gums of plant, microbial and/or synthetic origin, being between 1 and 4% by weight, preferably between 2 and 4% by weight, relative to the total weight of the granule.

This granule can have one or more of the features relative to the granules listed hereinabove in the description of the cosmetic composition according to the disclosure.

In particular, it can have a size, measured by screening, between 315 and 1250 μm.

It can furthermore contain 1 to 50% by weight, preferably 5 to 50% by weight and preferentially 5 to 40% by weight, relative to the total weight of the granule, of anionic surfactant(s).

In particular embodiments of the disclosure, the granule comprises a total amount of surfactants between 1 and 50% by weight relative to the total weight of said granule.

A further aspect of the disclosure relates to a method for preparing a disintegratable granule according to the disclosure, by wet granulation. This method comprises successive steps of:
preparing a mixture containing said at least one anionic surfactant, said at least one absorbent agent and said at least one thickening agent, in an amount between 1 and 4% by weight, preferably between 2 and 4% by weight, relative to the total weight of the granule,
adding a wetting liquid to this mixture,
forming a granule, for example by mechanical shearing or pulverisation,
and drying this granule, preferably so as to obtain a residual amount of wetting liquid less than or equal to 0.5% by weight relative to the total weight of the granule.

Preferably, the wetting liquid is of aqueous type. It preferably contains more than 50% by weight of water, preferably more than 80% by weight of water.

In specific implementations of the disclosure, the wetting liquid is formed solely of water. In alternative implementations, it comprises, besides water, one or more water-soluble cosmetically acceptable ingredients, such as water-soluble cosmetic active agents, for example a nettle root extract, and/or water-soluble emollients.

Preferably, the wetting liquid contains less than 2% by weight, preferably less than 1% by weight, and preferably 0% by weight, of polyol(s), in particular propylene glycol.

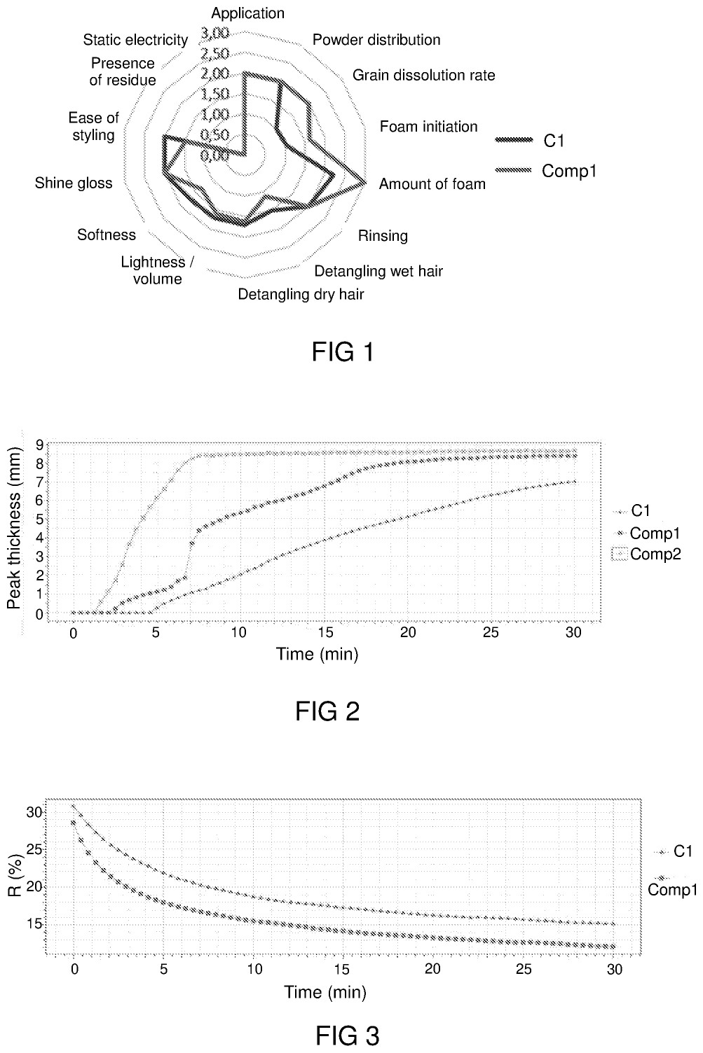

The features and advantages of the invention will become more apparent in the light of the examples of implementation hereinafter, provided merely by way of illustration and not restriction of the invention, with reference to FIGS. 1 to 3, wherein:

### Example 1: Formulation of a Composition According to the Disclosure

A cosmetic composition in dry form according to the disclosure has the composition indicated in Table 1 hereinafter.

TABLE 1
Cosmetic composition according to disclosure

| Ingredients | Amount (% by weight) |
|---|---|
| Sodium cocoyl isethionate | 5-50 |
| Coconut fatty acid | 0.5-5 |
| Corn starch | 1-70 |
| Rice starch | 1-70 |
| Hectorite | 2-10 |
| White clay (Kaolin) | 2-10 |
| Gum arabic | 0.5-2 |
| Xanthan gum | 0.5-2 |
| Dry bamboo extract | 0.5-2 |
| Glyceryl laurate | 0.6-1 |
| Perfume | 0.5-1 |
| Propylene glycol | 0-1 |
| Stinging Nettle root extract | 0-0.5 |
| Water | 0-0.5 |

### Example 2: Method for Obtaining Granules According to the Disclosure

The granules according to the disclosure are obtained by a granulation step, more specifically wet granulation.

The purpose of granulation is to convert crystallised or amorphous powder particles into more or less resistant and more or less porous solid aggregates referred to as granules or grains after calibration.

The advantages of the granules according to the disclosure, obtained by granulation, are multiple:
superior preservation of the homogeneity of the constituent mixture. Indeed, granulation will induce the appearance of bonds between the particles of the different constituents, these bonds helping obtain and maintain the homogeneity of the mixture;
good flow of the composition containing them. This improvement of the flow properties of the mixture could in particular be due to the increase in the density and size of the particles through the formation of a granule, and to the modification of the shape and the surface condition of these particles;
a reduction in dust generation compared to the same mixture in powder form. The granule form thus especially helps reduce cross-contamination problems and facilitates cleaning operations during the manufacture and packaging of the composition;
control of the dissolution/dispersion rate of the granule constituents in water. Indeed, the use of binders, such as the absorbent agent contained in the granules according to the disclosure, which are generally hydrophilic substances, during granulation, can result in an improvement in the wetting of the surfaces and thereby in particular improve the dissolution rate of the hydrophobic constituents in water.

Wet granulation consists of the agglomeration of powders using a wetting liquid. A distinction is made between the following steps:
mixing the raw materials: the purpose of this step is to distribute the constituents homogeneously;
manufacturing a binder solution and transferring to the granulator;
wetting the powders and mixing; the purpose of this step is to create bonds between the particles, which must be strong enough for the whole to withstand the granulation operation per se;
granulation, the purpose of this step being to mix powder and liquid to achieve the granular structure, by mechanical shearing or by pulverisation;
distributing wet granules on tray; this step is only present when subsequent oven drying is provided;
drying, this step making it possible to reduce the moisture content to a value adapted to preventing the degradation of the constituents and enabling the shaping of the granules;
calibration, for obtaining a good particle size distribution by a passing through a calibrated grid.

In order to ensure the conformity of the granules obtained, several inspection methods relating to several parameters can be used: particle size, apparent density, porosity, hardness, residual moisture, disintegration, dissolution.

In this example, the following specific procedure was used, for a batch size of 210 kg, in a 600 litre Lödige type mixer:
mix the powders forming the granules for 3 minutes (in this example, all the ingredients indicated in Table 1 above, except for hectorite, kaolin and perfume);
wet the powders by spraying 3×10 litres of water in 3×3 minutes while mixing;
dry the wet granules in a tray dryer oven at 75° C. until the targeted relative moisture percentage, preferably less than 4% as measured by a halogen dryer for 2 g for 10 minutes at 105° C.;
calibrate the dry grains on a 1.25 mm grid for example;

To the granules thus obtained, the other ingredients making up the cosmetic composition are added (in this example, hectorite powder, kaolin powder and perfume, the latter being absorbed on the hectorite and kaolin powders), and make the final mixture, the granules constituting more than 50% by volume of the composition.

### Example 3: Characterisation of the Cosmetic Compositions and Granules According to the Disclosure

The purpose of this study is to characterise different samples of the cosmetic composition according to the disclosure and of granules used in the composition thereof.

The techniques used are:
screening, which is used to determine the particle size distribution of the sample;
Densi-Tap™, which is used to determine the apparent density and the tap density of the powders and the granules (as per the standard ASTM D4164).
the Hall/Carney funnel flowmeter (Carney cone), which is used to characterise the flowability for each sample by determining the flow time.

The samples studied are as follows:
Composition comprising the ingredients indicated in Table 1 above in the form of separate powders (comparative example of composition not according to the disclosure);
Granules formed as described in example 2;
Final composition, according to the disclosure, containing these granules and the additional ingredients, as described in example 2.

### Analytical Techniques

### Particle Size Distribution by Screening

The particle size distribution was carried out by screening. The screens were adapted according to the samples. For the powder sample, five screen of sizes 25 μm, 53 μm, 75 μm, 150 μm and 315 μm, were selected. For the granule samples, five screen of sizes 75 μm, 150 μm, 315 μm, 600 μm and 1 mm, were selected. Screening was performed until a constant mass per size range was obtained.

### Apparent Density and Tap Density

Characterisation technique: compaction volume
Apparatus: Stampfvolumeter STAV 2003 JEL
Principle: The method consists of inducing the compaction of a powder placed in a standardised test tube, having it undergo a series of standardised shocks. The tap density is determined after 200 shocks. The density is calculated according to the formula: p=m/V, where m is the powder mass in g, and V the powder volume in cm³.

### Flowability

The evaluation of flowability according to the Carney cone method consists of determining the flow time of metallic powders through a calibrated funnel with a 5 mm opening. The measurements are made on 25 ml test samples. The flow time of the entire test sample is measured. The flow time is calculated on a mean of three tests per sample.

### Results

The results obtained for the particle size distribution by screening are shown in Tables 2 (for the powder mixture) and 3 (for the granules and the composition according to the disclosure).

TABLE 2
particle size distribution of the powder mixture

| | |
|---|---|
| Size | Particle size distribution (%) of powder form |

| | | |
|---|---|---|
| <25 | μm | 6.1 |
| 25-53 | μm | 25.4 |
| 53-75 | μm | 42.2 |
| 75-150 | μm | 22.8 |
| 150-315 | μm | 0.8 |
| >315 | μm | 2.7 |

The sample is powder form has a particle size distribution centred around 53-75 μm.

TABLE 3
particle size distribution of the granules and the
whole composition according to the disclosure

Particle size distribution (%)

| | | |
|---|---|---|
| Size | Granules | Whole composition |

| | | | |
|---|---|---|---|
| <75 | μm | 5.8 | 14.8 |
| 75-150 | μm | 5.5 | 6.4 |
| 150-315 | μm | 17.2 | 13.9 |
| 315-600 | μm | 32.3 | 28.9 |
| 600-1000 | μm | 30.7 | 28.8 |
| >1 | mm | 8.5 | 7.2 |

The granule samples have a dispersed particle size distribution centred around 315 μm to 1 mm. It should be noted that the “whole composition” sample consists of a greater amount of fine particles less than 75 μm in size than the “granules” sample. Approximately 80% of the whole formula is in the form of granules greater than 150 μm in size.

The apparent density and tap density results are shown in Table 4 hereinafter, these results having been obtained from the mean of three measurements.

TABLE 4
Characteristics of the powder mixture and
the granules according to the disclosure

| Samples | Apparent density (g/cm³) | Tap density (g/cm³) |
|---|---|---|
| Powder form | 0.41 ± 0.01 | 0.56 ± 0.01 |
| Granules | 0.66 ± 0.01 | 0.76 ± 0.01 |

The samples in granule form have a much higher apparent density than the non-granulated, powder mixture sample. The granule samples are also less sensitive to compaction, indicating lower cohesion and potentially superior flow.

The flowability results are shown in Table 5.

TABLE 5
Flowability results

Flowability (s)

| Tests | Powder form | Granules | Whole composition |
|---|---|---|---|
| No. 1 | No flow | 14.76 | 14.92 |
| No. 2 | | 14.28 | 14.16 |
| No. 3 | | 14.32 | 14.15 |
| Mean | | 14.45 | 14.41 |

The sample in mere powders mixture form did not flow through the system used. On the other hand, the samples in granule form had similar flow times of the order of around fifteen seconds.

In conclusion, the analyses carried out in this study demonstrated firstly that wet granulation was effective; indeed, converting from a powder form to a granule form helps increase flowability substantially. Secondly, approximately 80% of the whole composition is actually in the form of granules greater than 150 μm in size.

### Example 4: Consumer Test of a Cosmetic Composition According to the Disclosure

The purpose of this test is to characterise perceptions and impressions during the use of the composition according to the disclosure as prepared in example 2, and determine the strengths and weaknesses thereof under actual conditions of use.

The consumers are surveyed in France and rate the product at home.

96 consumers were recruited for these tests, they are 19 to 63 years of age (mean 39 years), 77% of them are women. The product is rated anonymously, over a 2-month test use period. Five rating dates are required: DO, D1, D30, D60 and D75.

The protocol for use is as follows:
Pour the composition in granule form into the palm of the hand moistened beforehand;
Apply to wet hair;
Massage to form a foam;
Leave on hair for 1 to 2 minutes;
Rinse.

For optimum results, it is required to use this composition once a week for 2 months.

Quantification of results: Rating on a scale of 0 to 10, where 0: dislike, and 10: like a lot, or 0: totally disagree, and 10: totally agree.

### Results

Rating the cosmetic composition according to the disclosure overall, the immediate rating score out of 10 is 6.97, it is 6.94 on D30, and attains 7.00 on D60.

For the criteria in respect of powder conversion to foam, the score on D30 for example attains 7.51 out of 10 with 81% of the responses in the 5 highest ratings (6 to 10/10).

In terms of the amount of foam, 60% of consumers find the amount to be sufficient.

Similarly for the amount of powder per use, 66% of consumers find it to be enough.

For the cleaning effectiveness of the composition according to the disclosure, the cleanliness score out of 10 attains 7.70 for an immediate effect, 7.62 on D30, 7.69 on D60 and 7.44 on D75. Approximately 50% of consumers give a rating of at least 8/10.

On the other hand, on the impression of having greasy hair, before using the composition according to the disclosure, the score out of 10 attains 5.67 and drops to 1.73 immediately after using the composition according to the disclosure, demonstrating the effectiveness of said composition.

Similar results are found when focusing on the scalp.

Finally, on the question of comparing the composition according to the disclosure to the usual product, 38% of consumers find it much better, 23% somewhat better, and merely 1% of consumers find it considerably worse.

This consumer test allowed to rate the cosmetic composition in dry form according to the disclosure qualitatively. It appears that it mixes well with water and that the granules disintegrate easily. It is easy to apply to the skin and hair. It has good foaming properties, the foam initiation is satisfactory, and an adequate and sufficiently abundant foam is obtained, and the foam quality is good, in particular the foam is creamy and abundant.

### Example 5: Comparisons with and without Thickening Agent

In this example, a cosmetic composition C1 according to the disclosure is compared to a comparative cosmetic composition Comp1 not according to the disclosure, with the same composition, but devoid of gum.

These compositions are obtained as described in example 2, with the constituents in the proportions indicated in Table 6 hereinafter.

TABLE 6

| Ingredient | C1 - amount (% w/w) | Comp1 - amount (% w/w) |
|---|---|---|
| Sodium cocoyl | 45 | 45 |
| isethionate | | |
| Corn starch | 35 | 37 |
| Rice starch | 16 | 16 |
| Gum arabic | 1 | — |
| Xanthan gum | 1 | — |
| Dry bamboo extract | 1 | 1 |
| Hair conditioner | 1 | 1 |

A sensory analysis, according to the following criteria, was carried out for each of these compositions, by applying the protocol described in example 4 above, on wet hair: application, powder distribution, grain dissolution rate, foam initiation, foam amount, rinsing, wet hair detangling, dry hair detangling, lightness/volume, softness, shine/gloss, ease of styling, presence of residue after rinsing, static electricity. The results obtained are shown in FIG. 1.

It is observed that, unlike composition Comp1, for the composition according to the disclosure C1:
foam initiation is slower—the conversion process occurs in steps: the powder is first converted into a creamy massage lotion with undissolved granules still present, then a complete conversion into foam is observed—the exfoliating action is accentuated at the start of use, relative to the comparative composition Comp1;
the amount of foam is smaller—this amount is nonetheless considered to be satisfactory by most users—so that a lesser amount of water is required for rinsing;
the sensation of softness is greater.

These advantages are not obtained at the cost of the expected beneficial effects for the hair, particularly in relation to ease of styling, which is even actually improved, particularly on the detangling aspect of wet hair. Furthermore, the hair appears to be stronger, without being weighed down.

The composition according to the disclosure C1 and the comparative composition not according to the disclosure Comp1 are furthermore subjected to a physical stability test. Another comparative composition not according to the disclosure, named Comp2, also devoid of gum as a thickening agent, is also subjected to this test. This composition Comp2 has the composition of example 3 of the document FR 3 068 243.

To this end, the following protocol is used:
in a cell for a Turbiscan® apparatus, weigh 0.05 g of composition and make up to 5 mL with distilled water.
seal the cell and shake 50 times to induce foam formation.
place the cell immediately in the Turbiscan® apparatus and run a scan analysis every 20 seconds for 30 minutes.

The Turbiscan® is an apparatus which sends a light beam to a sample present in a transparent cell, sweeping from the bottom of the sample upwards. It can then detect the luminous flux transmitted (passing through the sample) and backscattered (not passing through the sample). By repeating the measurement at an adapted frequency over time, the instrument makes it possible to track the stability of the sample.

Regarding the transmitted luminous flux, the measurement of the peak thickness as a function of time makes it possible to observe the rate of formation of the drainage phase in the bottom of the cell. Increased transmission means that light can pass through the sample, therefore, that there are no more bubbles in the bottom of the sample, and they have been replaced by a drainage liquid which allows light through, indicating foam destruction. The greater the slope of the curve, the faster the destabilisation of the sample.

The results obtained for the 3 samples tested are shown in FIG. 2. It is observed that the curve corresponding to the comparative composition Comp2 has a higher slope than the others, which shows that the drainage liquid appeared more quickly therein. The drainage phase also appeared much more quickly for the comparative composition Comp1 than for the composition according to the disclosure C1. These results clearly demonstrate that the foam obtained with the composition according to the disclosure C1 is much more stable than those obtained with the comparative compositions, its bubbles bursting more quickly. It is particularly advantageously very stable for the first 5 minutes, namely for the ideal duration of use of a keratin fibre cleansing composition. This stability ensures in particular optimal cleansing effectiveness.

Regarding backscatter, it also allows to determine the speed at which the bubbles burst in the centre of the cell. A high backscatter means that the bubbles do not allow light to pass through the sample; on the other hand, a lower backscatter means that there are no more bubbles, as light can pass through the cell.

The results obtained are shown in FIG. 3, for the composition according to the disclosure C1 and the comparative composition Comp1. It is observed that the slope of the curve corresponding to the latter is higher, indicating a poorer foam stability than for the composition according to the disclosure C1.

### Example 6: Formulation of a Composition According to the Disclosure—Anti-Dandruff Shampoo

A cosmetic composition in dry form according to the disclosure has the composition indicated in Table 7 hereinafter.

TABLE 7

| Ingredients | Amount (% by weight) |
|---|---|
| Sodium cocoyl isethionate | 25-50 |
| Corn starch | 30-50 |
| Aluminum silicate | 10-30 |
| Green clay | 2-10 |
| Gum arabic | 0.5-2 |
| Xanthan gum | 0.5-2 |
| Perfume | 0.5-1 |
| Hair conditioner | 0.5-2 |
| Dry Alpinia galanga extract | 0.3-1.0 |
| Water | 0-0.5 |

This composition is prepared as described in example 2.

It has similar properties to those of the composition in example 2 and composition C1 of example 5 above.

What is claimed is:

1. A cosmetic composition in dry form, suitable for being topically applied to the skin and/or keratin fibres, comprising at least 50% by volume of disintegratable granules having a size, measured by screening, between 150 and 2000 μm and containing a mixture of at least one anionic surfactant and at least one absorbent agent selected in the group consisting of clays, ceramic beads, calcium carbonate, titanium oxides, kaolin, aluminium silicates, starches, celluloses, sodium carbonate, Nylon microspheres and plant-based powders, said disintegratable granules further comprising at least one thickening agent selected in the group consisting of gums of plant, microbial and/or synthetic origin, the total amount of said thickening agent(s) in said composition being between 1 and 4% by weight relative to the total weight of the composition.

2. The cosmetic composition according to claim 1, comprising at least 75% by volume of said granules.

3. The cosmetic composition according to claim 1, wherein the size of said granules is between 315 and 1250 μm.

4. The cosmetic composition according to claim 1, wherein the total amount of said thickening agent(s) selected in the group consisting of gums of plant, microbial and/or synthetic origin, present in said disintegratable granules, is between 2 and 4% by weight relative to the total weight of the composition.

5. The cosmetic composition according to claim 1, containing an amount between 1 and 50% by weight, relative to the total weight of the composition, of anionic surfactant(s).

6. The cosmetic composition according to claim 5, containing an amount between 5 and 40% by weight, relative to the total weight of the composition, of anionic surfactant(s).

7. The cosmetic composition according to claim 1, wherein said anionic surfactant is selected in the group consisting of isethionic acid derivatives and salts thereof.

8. The cosmetic composition according to claim 1, containing a total amount of surfactants between 1 and 50% by weight relative to the total weight of the composition.

9. The cosmetic composition according to claim 1, wherein said absorbent agent is starch.

10. The cosmetic composition according to claim 1, wherein said granules further comprise at least one clay.

11. The cosmetic composition according to claim 1, further comprising, outside said granules, at least one exfoliating agent.

12. The cosmetic composition according to claim 1, wherein said granules contain an amount of polyol of less than 2% by weight, relative to the total weight of the granules.

13. The cosmetic composition according to claim 1, comprising a cosmetically active agent.

14. The cosmetic composition according to claim 1, packaged in single-dose form.

15. A method for washing and/or cosmetically treating keratin fibres, comprising a step of applying, to said damp keratin fibres, a cosmetic composition as defined in claim 1, followed by rinsing of said keratin fibres with water.

16. A method for washing and/or cosmetically treating the skin, comprising a step of applying, to said damp skin, a cosmetic composition as defined in claim 1, followed by rinsing of said skin with water.

17. (canceled)

18. A disintegratable granule suitable for being topically applied to an individual's skin and/or keratin fibres, having a size, measured by screening, between 150 and 2000 μm and a containing a mixture of at least one anionic surfactant and at least one absorbent agent selected in the group consisting of clays, ceramic beads, calcium carbonate, titanium oxides, kaolin, aluminium silicates, starches, celluloses, sodium carbonate, Nylon microspheres and plant-based powders, said granule containing at least one thickening agent selected in the group consisting of gums of plant, microbial and/or synthetic origin, the total amount of said thickening agent(s) in said granule being between 1 and 4% by weight relative to the total weight of the granule.

19. The granule according to claim 18, containing 1 to 50% by weight, relative to the total weight of the granule, of anionic surfactant(s).

20. The granule according to claim 18, containing a total amount of surfactants between 1 and 50% by weight relative to the total weight of said granule.

21. (canceled)

22. (canceled)
